(19) United States
(12) Patent Application Publication
(10) Pub. No.: US 2024/0108485 A1
(43) Pub. Date: Apr. 4, 2024

(54) Holding system, device, and method for holding tools and utensils

(71) Applicant: Michael Patrick Haluska, Sarasota, Fl (US)

(72) Inventor: Michael Patrick Haluska, Sarasota, Fl (US)

(21) Appl. No.: 18/129,291

(22) Filed: Mar. 31, 2023

Related U.S. Application Data
(60) Provisional application No. 63/411,336, filed on Sep. 29, 2022.

(51) Int. Cl. A61F 4/00

(52) U.S. Cl. CPC A61F 4/00

(57) ABSTRACT

A holding device includes a finger connector piece, including a finger connector body with a central finger aperture and a side slit; and a utensil connector piece, including a utensil connector body with a slot-shaped central utensil aperture, configured with a central opening and outer and inner pluralities of lateral ridges on opposing sides of the central aperture; such that, when the finger connector piece is mounted on a finger, the utensil connector piece is configured to provide a primary grip of a utensil mounted in the utensil connector piece, such that the hand of the user is free to provide fine motor control of the utensil.

### CROSS-REFERENCE TO RELATED APPLICATIONS

This application claims the benefit of U.S. Provisional Application No. 63/411,336, filed Sep. 29, 2022.

### FIELD OF THE INVENTION

The present invention relates generally to the field of utensil accessories, and more particularly to methods and systems for utensil holding devices.

### BACKGROUND OF THE INVENTION

Many people struggle with holding everyday objects and may develop pain and injuries from repetitive use of tools or utensils. This can particularly be the case for older individuals or people that suffer from arthritis or other disorders causing reduced finger dexterity and control.

Furthermore, difficulty of handling everyday tools and utensils can lead to anxiety or depression, caused by the frustration and emotional upset that arises from experiencing a worsening loss of finger grip control.

However, there are only few available devices and systems available for improving finger grip, and these tend be more oriented towards guiding fingers of a user, rather than providing a direct holding grip of the tool or utensil.

As such, considering the foregoing, it may be appreciated that there continues to be a need for novel and improved devices and methods for improving finger grip of tools and utensils.

### SUMMARY OF THE INVENTION

The foregoing needs are met, to a great extent, by the present invention, wherein in aspects of this invention, enhancements are provided to the existing model of for improving finger grip of tools and utensils.

In an aspect, a holding device can include:
a) a finger connector piece, which can include:
   i. a finger connector body, which can include a central finger aperture, which can be circular;
   such that the finger connector body is configured to be mounted on a finger of a user, such that the finger penetrates through the central finger aperture; and
b) a utensil connector piece, which can include:
   i. a utensil connector body, which can include a central utensil aperture;
   such that the utensil connector body is configured to be mounted on an elongated portion of a utensil, such that the elongated portion penetrates through the central utensil aperture;
   such that an inner end of the finger connector body is connected to an inner end of the utensil connector body;
   such that, when the finger connector piece is mounted on the finger, the utensil connector piece is configured to provide a primary grip of the utensil, such that the hand of the user is free to provide fine motor control of the utensil.

In a related aspect, the inner end of the finger connector body can be connected to an inner end of the utensil connector body with a longitudinal connection angle of about 25-30 degrees or in a range of 20-35, 15-40, 10-45, or 5-50 degrees.
in another related aspect, the central finger aperture can be configured with an inner slit, which protrudes from an inner side of the central finger aperture toward the utensil connector piece, such that the inner slit can enable enhanced flexibility of the central finger aperture.
in yet another related embodiment, the central utensil aperture can be slot-shaped, such that the central utensil aperture is configured with an elongated opening of a uniform or variable height, which can be in a range of 0.5-5 mm (i.e., about 0.0195-0.1669 inches).
in yet a related aspect, the central utensil aperture can be configured with a central opening, which is wider than inner and outer parts of the central utensil aperture, wherein the central opening can be substantially circular, such that the central opening can aid in holding a utensil, such that upper and lower edges of the utensil can rest in the central opening.
in yet a related embodiment, the central utensil aperture can be configured with:
a) an outer plurality of outer lateral ridges, positioned on an outer side of the central opening; and
b) an inner plurality of inner lateral ridges, positioned on an inner side of the central opening.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. In addition, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1A is a perspective view of a holding device in use holding a fork, according to an embodiment of the invention.

FIG. 18 is a perspective view of a holding device in use holding a pencil, according to an embodiment of the invention.

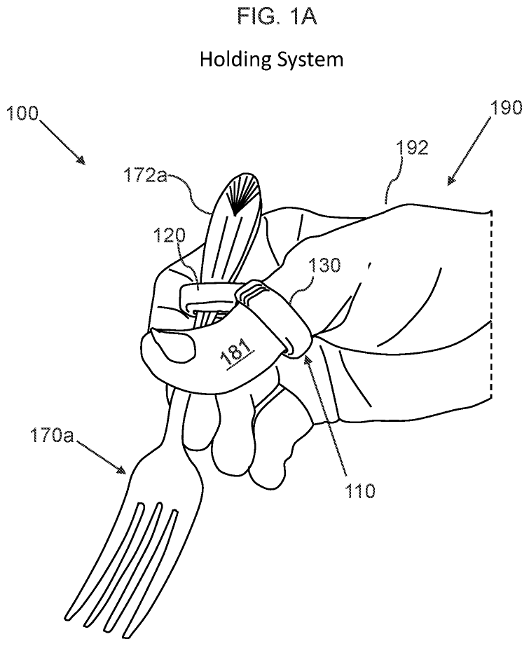

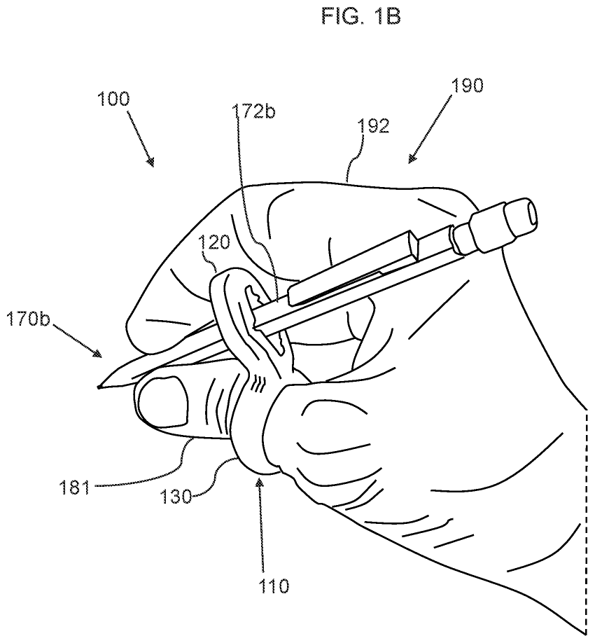

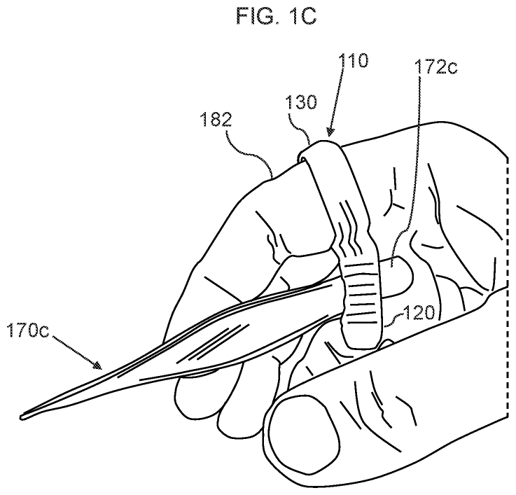

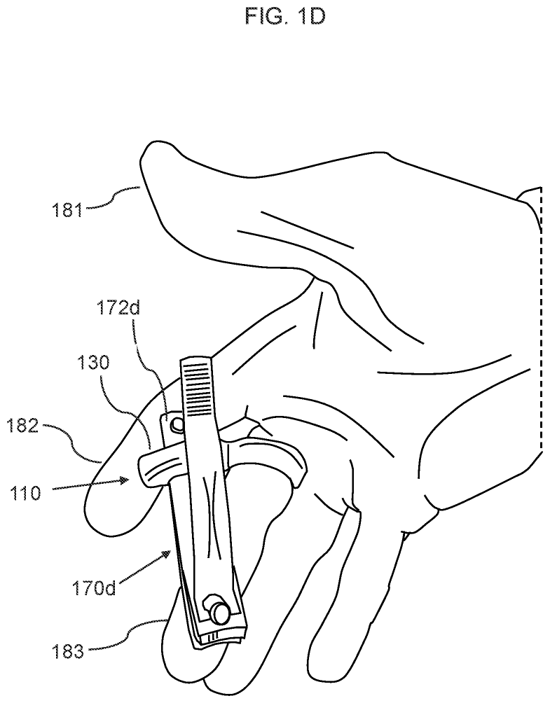

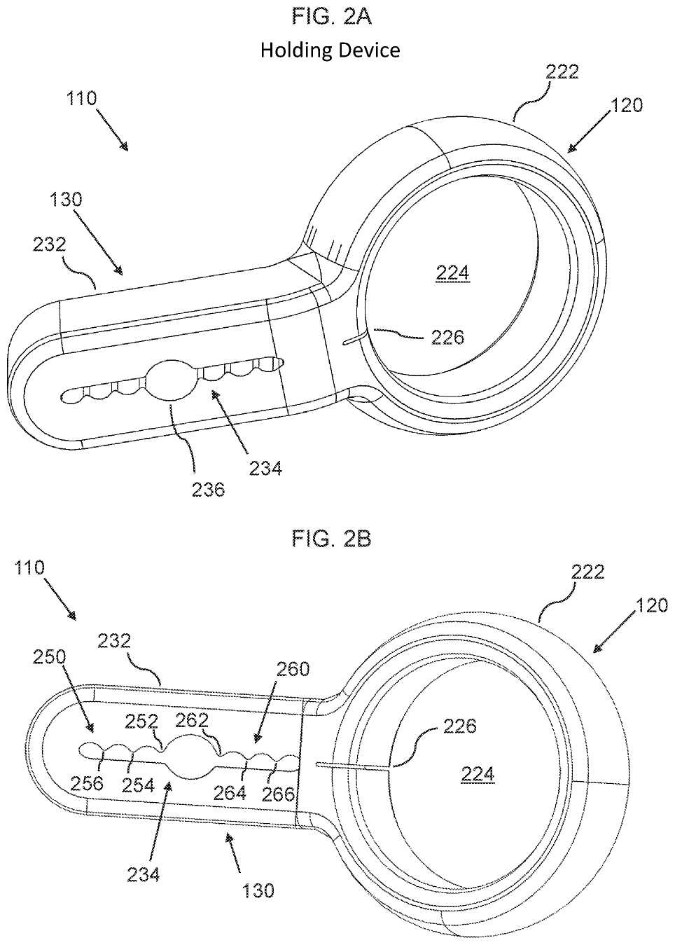

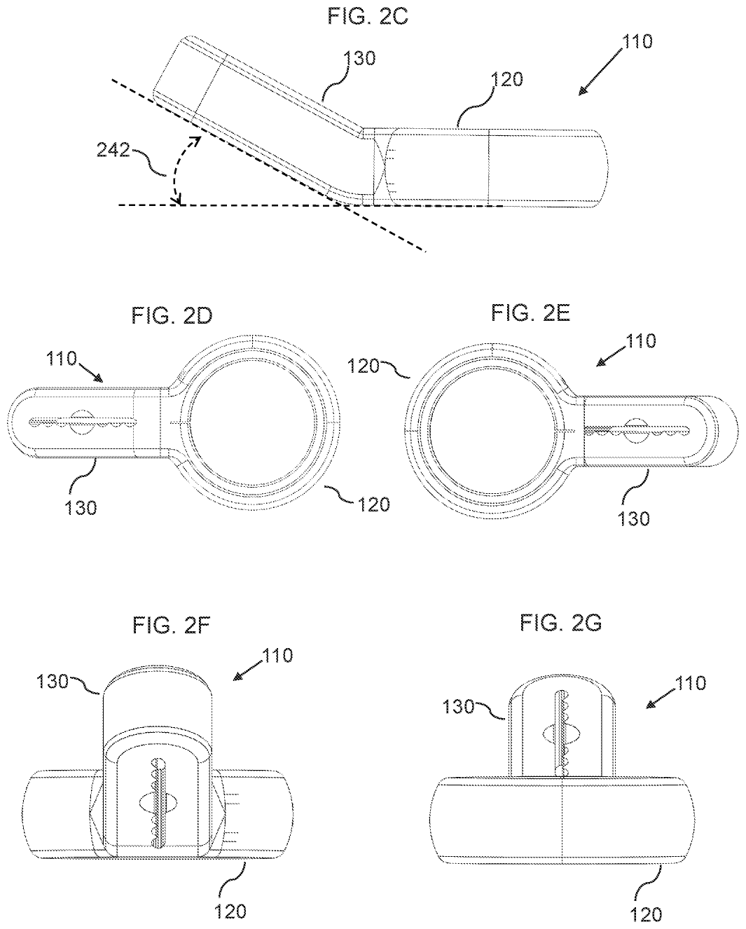

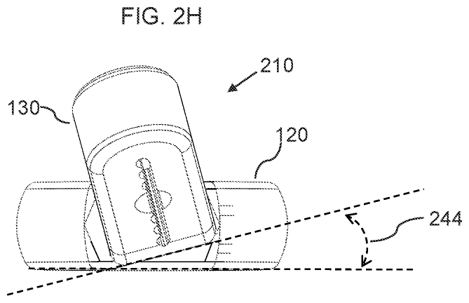

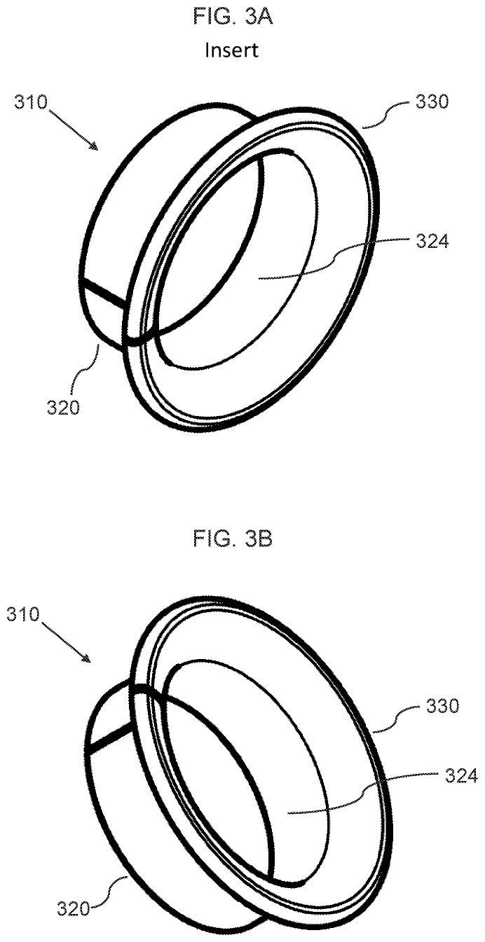

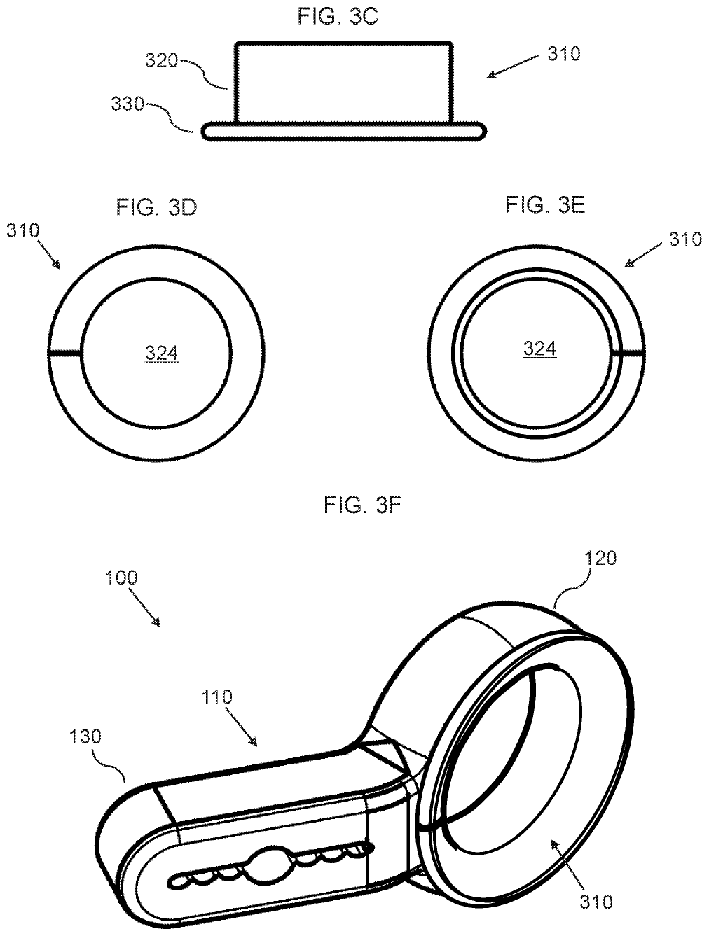

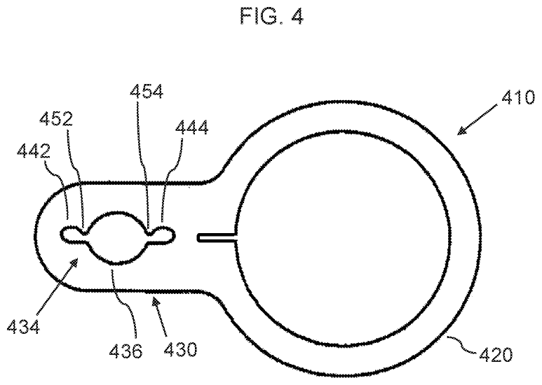

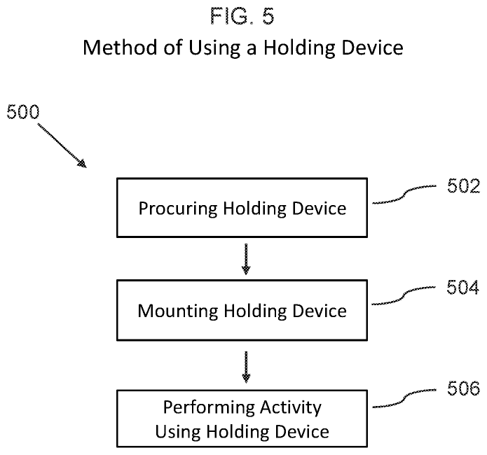

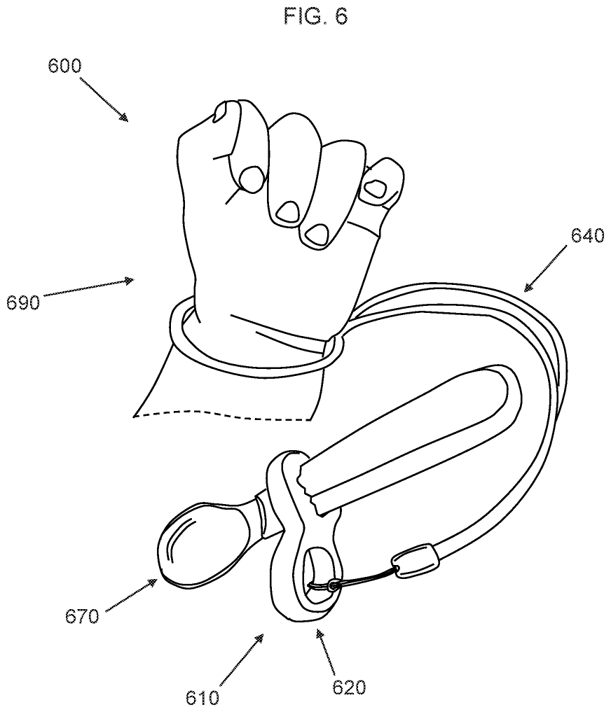

FIG. 1C is a perspective view of a holding device in use holding a tweezer, according to an embodiment of the invention.

FIG. 1D is a perspective view of a holding device in use holding a nailcutter, according to an embodiment of the invention.

FIG. 2A is a top right perspective view of a holding device, according to an embodiment of the invention.

FIG. 2B is a top left perspective view of a holding device, according to an embodiment of the invention.

FIG. 2C is a left side elevational view of a holding device, according to an embodiment of the invention.

FIG. 2D is a top plan view of a holding device, according to an embodiment of the invention.

FIG. 2E is a bottom plan view of a holding device, according to an embodiment of the invention.

FIG. 2F is a front elevational view of a holding device, according to an embodiment of the invention.

FIG. 2G is a rear elevational view of a holding device, according to an embodiment of the invention.

FIG. 2H is a front elevational view of a holding device, according to an embodiment of the invention.

FIG. 3A is a top perspective view of an insert piece, according to an embodiment of the invention.

FIG. 3B is a top perspective view of an insert piece, according to an embodiment of the invention.

FIG. 3C is a side elevational view of an insert piece, the left side, front, and rear elevational views being identical thereto, according to an embodiment of the invention.

FIG. 3D is a top plan view of an insert piece, according to an embodiment of the invention

FIG. 3E is a bottom plan view of an insert piece, according to an embodiment of the invention

FIG. 3F is a top perspective view of a holding device with an insert piece mounted, according to an embodiment of the invention.

FIG. 4 is a top plan view of a holding device, according to an embodiment of the invention.

FIG. 5 is a flowchart illustrating steps that may be followed, in accordance with one embodiment of a method or process of using a holding device.

FIG. 6 is a perspective view of a holding system with a holding device secured by lanyard, according to an embodiment of the invention.

### DETAILED DESCRIPTION

Before describing the invention in detail, it should be observed that the present invention resides primarily in a novel and non-obvious combination of elements and process steps. So as not to obscure the disclosure with details that will readily be apparent to those skilled in the art, certain conventional elements and steps have been presented with lesser detail, while the drawings and specification describe in greater detail other elements and steps pertinent to understanding the invention.

The following embodiments are not intended to define limits as to the structure or method of the invention, but only to provide exemplary constructions. The embodiments are permissive rather than mandatory and illustrative rather than exhaustive.

In the following, we describe the structure of an embodiment of a holding system 100 with reference to FIG. 1A, in such manner that like reference numerals refer to like components throughout; a convention that we shall employ for the remainder of this specification.

In an embodiment, as shown in FIGS. 1A-1C and 2A-2B, a holding device 110 can include:
a) a finger connector piece 120, which can include:
   i. a finger connector body 222, which can include a central finger aperture 224, which can be circular;
   such that the finger connector body 222 is configured to be mounted on a finger 181, 182, 183 of a hand 192 of a user 190, such that the finger penetrates through the central finger aperture 224; and
b) a utensil connector piece 130, which can include:
   i. a utensil connector body 232, which can include a central utensil aperture 234;
   such that the utensil connector body 232 is configured to be mounted on an elongated portion 172a, 172b, 172c, 172d of a utensil 170a, 170b, 170c, 170d, such that the elongated portion 172a, 172b, 172c penetrates through the central utensil aperture 234;
   such that an inner end of the finger connector body 222 is connected to an inner end of the utensil connector body 232;
   such that, when the finger connector piece 120 is mounted on the finger 181, 182, 183 the utensil connector piece 130 is configured to provide a primary grip of the utensil 170a, 170b, 170c, 170d, such that the hand of the user is free to provide fine motor control of the utensil 170a, 170b, 170c, 170d;
   whereby the holding device 110 is configured to provide a naturalistic holding method to enable improved grasping, holding, and control of the utensil 170a, 170b, 170c, 170d.

In various related embodiments, the tool 170a, 170b, 170c, 170d (which can be a utensil 170a, 170b, 170c, 170d) can include a fork 170a, or other handheld silverware/eating utensils, a pencil 170b, a pen or other writing instrument, a tweezer 170c or similar hand tool, a toothbrush, a paintbrush, a nail clipper 170d, a nail file, a cutting knife, a crochet or knitting tool, or another type of handheld tool or utensil.

In a related embodiment, as shown in FIG. 2C, the inner end of the finger connector body 222 can be connected to an inner end of the utensil connector body 232 with a longitudinal connection angle 242 of about 25-30 degrees (as shown) or in a range of 20-35, 15-40, 10-45, 5-50 degrees, or larger, such that the longitudinal connection angle 242 can be configured to provide an improved ergonomic positioning of the utensil 170a, 170b, 170c, 170d, for comfortable use of the holding device 110.

In a related embodiment, as shown in FIG. 2H, the inner end of the finger connector body 222 can be connected to an inner end of the utensil connector body 232 with an axial rotational angle 244 in a range of about 10-20 degrees or in a range of 5-25, 2-30, 1-45, 1-50 degrees, or larger. The axial rotational angle 244 can be positive for a left-leaning axial rotational angle (as shown), or can be negative for a right-leaning axial rotational angle, with similar negative ranges; such that the axial rotational angle 244 can be configured to provide an improved ergonomic positioning of the utensil 170a, 170b, 170c, 170d, for comfortable use of the holding device 110.

In another related embodiment, as shown in FIGS. 2A-2B, the finger connector body 222 can be configured with an inner slit 226, which protrudes from an inner side of the central finger aperture 224 toward the utensil connector piece 130, such that the inner slit 226 can enable enhanced flexibility of the central finger aperture 224.

In yet another related embodiment, the central utensil aperture 234 can be slot-shaped, such that the central utensil aperture 234 is configured with an elongated opening of a uniform or variable height, which can be in a range of 0.5-5 mm (i.e., about 0.0195-0.1669 inches).

In a further related embodiment, the holding device 110 can be made from a flexible resilient material, such as a rubber material, which can be a natural or synthetic rubber material, such as a silicone gel, such that the slot-shaped central utensil aperture 234 is configured to be flexible, as shown in FIG. 1B, such that the slot-shaped central utensil aperture 234 is configured to adapt to different utensil sizes.

in yet a related embodiment, the central utensil aperture 234 can be configured with a central opening 236, which is wider than inner and outer parts 260, 250 of the central utensil aperture 234 (wherein each of the inner part 260 and the outer part 250 can be elongated and substantially slot-shaped), wherein the central opening 236 can be substantially circular, such that the central opening 236 can aid in holding a utensil with a circular handle or utensil with a center protrusion on handle, such that an elongated portion 172a, 172b, 172c handle portion of the utensil 170a, 170b, 170c, 170d is secured in the central opening 236, such that an upper edge and a lower edge of the utensil handle can each rest in the central opening 236.

In yet a related embodiment, as shown in FIGS. 2A and 2B, the central utensil aperture 234 can be configured with an entire plurality of lateral ridges 252, 254, 256, 262, 264, 266 on a first inner elongated side of the central utensil aperture 234, such that the lateral ridges can aid in ensuring a grip of an elongated portion 172a, 172b, 172c of a utensil 170a, 170b, 170c, 170d, wherein the entire plurality of lateral ridges can include:
a) an outer plurality of outer lateral ridges 252, 254, 256, which penetrate laterally from a top side of the utensil connector body 232 to a bottom side of the utensil connector body 232 on an outer side of the central opening 236 (i.e., in the outer portion 250); and
b) an inner plurality of inner lateral ridges 262, 264, 266, which penetrate laterally from a top side of the utensil connector body 232 to a bottom side of the utensil connector body 232 on an inner side of the central opening 236 (i.e., in the inner portion 260).

In a related embodiment, the entire plurality of lateral ridges 252, 254, 256, 262, 264, 266 can be configured on a first inner elongated side of the central utensil aperture 234, such that the first inner elongated side is ribbed, and such that an opposing second inner elongated side of the central utensil aperture 234 can be flat/smooth (on both sides of the central opening 236 if present). In other related embodiments, the first and second inner elongated sides can both be ribbed, or they can both be flat/smooth (in both cases optionally including a central opening), in order to adjust a grip of an elongated portion 172a, 172b, 172c, 172d of a utensil 170a, 170b, 170c, 170d positioned in the central utensil aperture 234 of the utensil connector body 232.

In another related embodiment, as shown in FIG. 4, a holding device 410 can be configured with a finger connector piece 420 connected to a relatively short utensil connector piece 430, wherein a central utensil aperture 434 can be configured with first and second short side openings 442, 444, each with a lateral ridge 452, 454, on either side of a central opening 436. The short side openings 442, 444 (which can also be called expansion slots) can facilitate flexibility of the central opening 436. The relatively short utensil connector piece 430 can be particularly helpful in use with a pen, pencil, crochet hook, or similar tools requiring a relatively close holding configuration.

In further related embodiments, a baby variant of the holding device 410 can be configured with a small central opening 436, that is adapted to a size of finger of a baby user 190. Such a baby variant of the holding device 410 can further be made of a softer material (i.e. with a smaller specified Durometer) than a holding device 110, 410 made for adult use. Baby variant of the of the holding device 410 may be used with a different gripping style when used by baby users 190 compared to the adult gripping style as described herein and shown in FIGS. 1A-1D. Baby users may grip with a closed fist, such as when holding a club or a stick, and wherein the baby variant of the holding device 410 is mounted on a thumb 181 of a hand 192 of the baby 190.

In an embodiment, as illustrated in FIG. 5, a method 500 of using a holding device 110, 410, can include:
a) Procuring 502 a holding device 110, 410, wherein a user 190 procures a holding device 110;
b) Mounting 504 the holding device 110, 410, wherein a finger connector body 222 of the holding device 110, 410 is mounted on a finger 182 of the user 190, such that the finger 182 penetrates through a central finger aperture 224 of the finger connector body 222; and wherein the utensil connector body 232 is mounted on an elongated portion 172a, 172b, 172c, 172d of a utensil 170a, 170b, 170c, 170d, such that the elongated portion 172a, 172b, 172c, 172d penetrates through the central utensil aperture 234; and
c) Performing 506 an activity using the holding device 110, 410 with the connected utensil 170a, 170b, 170c, 170d.

In related embodiments, as shown in FIGS. 1A-1D, the holding device 110, 410 can be mounted on different fingers of hand to support different uses with different tools, including, as shown, different actions that can be facilitated by varying the phalanges used. In some cases, the holding device 110, 410 may be mounted on any phalange of a finger or toe, for example for use by physically handicapped persons and/or related to particular use cases.

In various further related embodiments and related methods of use, as shown in FIGS. 1A-1D, the holding device 110, 410 can be mounted on a first digit 181, second digit 182, third digit 183, fourth digit, or fifth digit of a right or left hand 192 of a user 190.

In some embodiments and related methods of use, the holding device 110, 410 can be used in a “direct” manner” to support holding a utensil, such as a fork 170a, pencil 170b, or tweezer 170c.

In other embodiments and related methods of use, as shown in FIG. 1D, the holding device 110, 410 can be used in a “indirect” manner”, wherein one digit 182 is holding a supported utensil, and another digit 181 is manipulating the supported item 170d (such as a nail clipper 170d), wherein the holding device 110, 410 is mounted on yet another finger 183. FIG. 1D shows such an indirect use of a nail clipper 170d mounted on a third digit 183 of a hand 192 of a user 190.

In another related embodiment, as shown in FIGS. 3A-3F, the holding system 100 can further include:
a) an adaptor insert 310, which can include:
   i. a ring insert 320, comprising an inner finger aperture 324; and
   ii. an outer flange 330, which is connected to an outer circumference of the ring insert 320, wherein the outer flange 330 is configured to provide a smooth transition from an inner surface of the outer flange 330 to the inner surface of the ring insert 320;
   such that the ring insert is configured to be removably positioned inside the central finger aperture 224 of the finger connector body 222 (of the finger connector piece 120), in order to adapt the holding device 110 for use with smaller fingers 181, 182, 183.

In a further related embodiment, the holding system 100 can include a plurality of adaptor inserts 310, with different inner diameters of the ring insert 320 to accommodate different/smaller fingers 181, 182, 183 and/or different/smaller sizes of fingers 181, 182, 183.

In another related embodiment, as shown in FIG. 6, the holding system 100, 600 can further include:
a) a lanyard 610;
wherein a first end of the lanyard 640 can be configured to be detachably connectable to the holding device 110, 410, 610 and wherein a second end of the lanyard 640 can be configured to be detachably connectable to a wrist of the user 190, 690, whereby the lanyard can aid in retaining the holding device 110, 410 with an attached utensil 670, if the user 690 loses a grip of the holding device 110, 410. The holding system 600 with lanyard 610 can particularly be useful for use with babies 690 (as shown in FIG. 6), juveniles, elderly users, or other users that may be more prone to losing a grip of the holding device 110, 410, 610. As shown, the first end of the lanyard 640 can be attached (such as looped around) the finger connector piece 620.

Thus, in an alternative description of related embodiments, the holding device 110 can include:
a) the ½″ thick thumb ring with the ½″ thick straight protrusion that holds forks and spoons via its flat slot cut-out member and holds various circumference artist brushes, knitting needles, crochet hooks, writing implements, etc. within its circular cut-out at the center of its flat slot cut-out;
b) Note that the straight protrusion is fully supported by the thumb ring such that the item inserted into and carried by the straight protrusion is not held by the user, yet the object is able to be fully manipulated by the user. The thumb ring fully supports the item inserted into and carried by the straight protrusion;
c) The thickness of the protrusion is a variable that allows for support of the object (i.e., a tool or utensil) inserted into the straight protrusion;
d) The width of the thumb ring allows for and provides the stability and transfer of force and weight necessary to fully carry the objects inserted into the slot and hole within the straight protrusion;
e) The Durometer of the material used for the product can be configured to ensure user comfort as well as flexibility to aid in manipulating objects attached to the device;
f) A connection bend/angle between the ring element and the straight protrusion element, functions as an aid to proper manipulation; and
g) Additionally, ridges contained within the flat slot cut-out help retain from slippage and allow for fine adjustment of objects inserted therein;
such that the only product that the holding device 110 allows for a 100% ‘Naturalistic’ holding and grasping regime; and thereby enables naturalistically grasping and holding an object in one's hand so that the object being held is held by the device not the individual's grasping or/and holding powers.

In an embodiment, a retention apparatus 110 for aiding users 190 in at least one primary activity using a tool or utensil, can include a body formed of at least an elastomeric material, wherein the body comprises:
a) a user ring portion; and
b) a straight tool protrusion portion, which includes a lengthwise slot, which is cut out in the center of the straight tool protrusion portion, wherein the lengthwise slot includes a plurality of fine motor control anti-slip ridges along a width of the lengthwise slot and a center hole which is configured with a diameter that is larger than the spaces formed by said ridges;
wherein the user ring portion and the straight tool protrusion are joined together in an adjacent and slightly angular manner;
wherein the user ring portion is adapted to encircle a human hand digit and the straight tool protrusion portion is adapted to support a tool that inserts into the lengthwise slot, wherein the tool is fully supported and manipulatable by the user without the user having direct contact with the tool;
wherein a width of the user ring portion is configured to enable tool stability and transfer of force and weight from the straight tool protrusion portion to the user ring portion.

Here has thus been described a multitude of embodiments of the holding device 110, and systems 100 and methods 500 related thereto, which can be employed in numerous modes of usage.

The many features and advantages of the invention are apparent from the detailed specification, and thus, it is intended by the appended claims to cover all such features and advantages of the invention, which fall within the true spirit and scope of the invention.

Many such alternative configurations are readily apparent and should be considered fully included in this specification and the claims appended hereto. Accordingly, since numerous modifications and variations will readily occur to those skilled in the art, the invention is not limited to the exact construction and operation illustrated and described, and thus, all suitable modifications and equivalents may be resorted to, falling within the scope of the invention.

What is claimed is:

1. A holding device, comprising:
a) a finger connector piece, comprising:
a finger connector body, comprising a central finger aperture;
such that the finger connector body is configured to be mounted on a finger of a hand of a user, such that the finger penetrates through the central finger aperture; and
b) a utensil connector piece, comprising:
a utensil connector body, comprising a central utensil aperture;
such that the utensil connector body is configured to be mounted on an elongated portion of a utensil, such that the elongated portion, penetrates through the central utensil aperture;
such that an inner end of the finger connector body is connected to an inner end of the utensil connector body; and
whereby, when the finger connector piece is mounted on the finger, the utensil connector piece is configured to provide a primary grip of the utensil, such that the hand of the user is free to provide fine motor control of the utensil.

2. The holding device of claim 1, wherein the inner end of the finger connector body is connected to the inner end of the utensil connector body with a longitudinal connection angle in a range of 15-40 degrees.

3. The holding device of claim 1, wherein the inner end of the finger connector body is connected to the inner end of the utensil connector body with an axial rotational angle of 5-25 degrees.

4. The holding device of claim 1, wherein the central utensil aperture is slot-shaped, such that the central utensil aperture is configured with an elongated opening.

5. The holding device of claim 1, wherein the holding device is made from a flexible resilient material, such that the central utensil aperture is configured to be flexible, such that the central utensil aperture is configured to adapt to different utensil sizes.

6. The holding device of claim 1, wherein the central utensil aperture is configured with a central opening, which is wider than inner and outer parts of the central utensil aperture, such that the central opening is configured to aid in holding the utensil, such that the elongated portion of the utensil is secured in the central opening.

7. The holding device of claim 6, wherein the central utensil aperture is configured with an entire plurality of lateral ridges, comprising:
a) an outer plurality of outer lateral ridges, which penetrate laterally from a top side of the utensil connector body to a bottom side of the utensil connector body on an outer side of the central opening; and
b) an inner plurality of inner lateral ridges, which penetrate laterally from the top side of the utensil connector body to the bottom side of the utensil connector body on an inner side of the central opening.

8. The holding device of claim 7, wherein the entire plurality of lateral ridges is configured on a first inner elongated side of the central utensil aperture, such that the first inner elongated side is ribbed, and such that an opposing second inner elongated side of the central utensil aperture is flat.

9. The holding device of claim 1, wherein the finger connector body is configured with an inner slit, which protrudes from an inner side of the central finger aperture toward the utensil connector piece, such that the inner slit enables enhanced flexibility of the central finger aperture.

10. A holding system, comprising a holding device, comprising:
a) a finger connector piece, comprising:
a finger connector body, comprising a central finger aperture;
such that the finger connector body is configured to be mounted on a finger of a hand of a user, such that the finger penetrates through the central finger aperture; and
b) a utensil connector piece, comprising:
a utensil connector body;
such that the utensil connector body is configured to be mounted on an elongated portion of a utensil;
such that an inner end of the finger connector body is connected to an inner end of the utensil connector body; and
whereby, when the finger connector piece is mounted on the finger, the utensil connector piece is configured to provide a primary grip of the utensil, such that the hand of the user is free to provide fine motor control of the utensil.

11. The holding system of claim 10, further comprising:
an adaptor insert, comprising:
a ring insert, comprising an inner finger aperture; and
such that the ring insert is configured to be removably positioned inside the central finger aperture of the finger connector body of the finger connector piece.

12. The holding system of claim 11, wherein the adaptor insert further comprises:
an outer flange, which is connected to an outer circumference of the ring insert, wherein the outer flange is configured to provide a smooth transition from an inner surface of the outer flange to an inner surface of the ring insert.

13. The holding system of claim 10, further comprising:
a lanyard;
wherein a first end of the lanyard is configured to be detachably connectable to the holding device, and wherein a second end of the lanyard is configured to be detachably connectable to a wrist of the user, whereby the lanyard is configured to aid in securing the holding device, if the user loses a grip of the holding device.

14. A method of using a holding device, comprising:
a) mounting the holding device, wherein a finger connector body of the holding device is mounted on a finger of a user, such that the finger penetrates through a central finger aperture of the finger connector body; and wherein a utensil connector body of the holding device is mounted on an elongated portion of a utensil, such that the elongated portion penetrates through a central utensil aperture of the finger connector body; and
b) performing an activity using the holding device with the utensil connected to the holding device;
wherein the holding device comprises:
a finger connector piece, comprising:
the finger connector body, comprising the central finger aperture;
such that the finger connector body is configured to be mounted on the finger of a hand of the user, such that the finger penetrates through the central finger aperture; and
a utensil connector piece, comprising:
the utensil connector body, comprising the central utensil aperture;
such that the utensil connector body is configured to be mounted on the elongated portion of the utensil, such that the elongated portion, penetrates through the central utensil aperture;
such that an inner end of the finger connector body is connected to an inner end of the utensil connector body; and
whereby, when the finger connector piece is mounted on the finger, the utensil connector piece is configured to provide a primary grip of the utensil, such that the hand of the user is free to provide fine motor control of the utensil.

15. The method of claim 14, wherein the inner end of the finger connector body is connected to the inner end of the utensil connector body with a longitudinal connection angle in a range of 15-40 degrees.

16. The method of claim 14, wherein the inner end of the finger connector body is connected to the inner end of the utensil connector body with an axial rotational angle of 5-25 degrees.

17. The method of claim 14, wherein the central utensil aperture is configured with a central opening, which is wider than inner and outer parts of the central utensil aperture, such that the central opening aids in holding the utensil, such that the elongated portion of the utensil is secured in the central opening.

18. The method of claim 17, wherein the central utensil aperture is configured with an entire plurality of lateral ridges, comprising:
a) an outer plurality of outer lateral ridges, which penetrate laterally from a top side of the utensil connector body to a bottom side of the utensil connector body on an outer side of the central opening; and
b) an inner plurality of inner lateral ridges, which penetrate laterally from the top side of the utensil connector body to the bottom side of the utensil connector body on an inner side of the central opening.

19. The method of claim 18, wherein the entire plurality of lateral ridges is configured on a first inner elongated side of the central utensil aperture, such that the first inner elongated side is ribbed, and such that an opposing second inner elongated side of the central utensil aperture is flat.

20. The method of claim 14, wherein the finger connector body is configured with an inner slit, which protrudes from an inner side of the central finger aperture toward the utensil connector piece, such that the inner slit enables enhanced flexibility of the central finger aperture.
